(19) United States
(12) Patent Application Publication
(10) Pub. No.: US 2009/0062886 A1
(43) Pub. Date: Mar. 5, 2009

(54) SYSTEMS AND METHODS FOR DELIVERING ELECTRICAL ENERGY IN THE BODY

(75) Inventors: Robert C. O'Handley, Andover, MA (US); Jiankang Huang, Arlington, MA (US); Jesse Simon, Jamaica Plain, MA (US); Kevin O'Handley, Haverhill, MA (US); Hariharan Sundrum, Brookline, MA (US)

Correspondence Address: RISSMAN JOBSE HENDRICKS & OLIVERIO, LLP, 100 Cambridge Street, Suite 2101, BOSTON, MA, 02114, US

(73) Assignee: Ferro Solutions, Inc., Woburn, MA (US)

(21) Appl. No.: 12/240,368

(22) Filed: Sep. 29, 2008

Related U.S. Application Data
(63) Continuation-in-part of application No. 11/734,181, filed on Apr. 11, 2007.
Application No. 11/734,181 is a continuation-in-part of application No. 11/652,272, filed on Jan. 11, 2007.
Application No. 11/652,272 is a continuation-in-part of application No. 10/730,355, filed on Dec. 8, 2003, now abandoned.
(60) Provisional application No. 60/976,030, filed on Sep. 28, 2007.
(60) Provisional application No. 60/791,004, filed on Apr. 11, 2006.
(60) Provisional application No. 60/790,921, filed on Apr. 11, 2006.
(60) Provisional application No. 60/431,487, filed on Dec. 9, 2002.

(51) Int. Cl. A61N 1/378

(52) U.S. Cl. 607/51; 607/2

(57) ABSTRACT

Small implantable magnetostrictive-electroactive (ME) device for delivering electrical energy to surrounding tissue. The wireless ME device is activated by a changing magnetic field from an externally applied alternating magnetic field source. The ME device provides a means for stimulating a nerve, tissue or internal organ with direct electrical current, such as relatively low-level direct current for temporary or as needed therapy. The field source (e.g. small coil antenna) may be a hand-held device or affixed to the wearer's skin, clothing or accessories. The ME implant may be configured as pellets which are small enough to be implanted through a surgical needle. In one embodiment, the wireless energy transmission system can be used for stimulating bone growth.

### RELATED APPLICATIONS

This application claims priority to U.S. Provisional Application No. 60/976,030 filed Sep. 28, 2007 and is a continuation in-part of U.S. application Ser. No. 11/734,181 filed Apr. 11, 2007, which claims the benefit of priority to U.S. Provisional Application No. 60/791,004, filed Apr. 11, 2006, and is a continuation-in-part of U.S. application Ser. No. 11/652,272, filed Jan. 11, 2007, which claims the benefit of priority to U.S. Provisional Application No. 60/758,042, filed Jan. 11, 2006, and U.S. Provisional Application No. 60/790,921, filed Apr. 11, 2006, and is a continuation-in-part of U.S. application Ser. No. 10/730,355, filed Dec. 8, 2003, which claims the benefit of priority to U.S. Provisional Application No. 60/431,487, filed Dec. 9, 2002, the disclosures of which are incorporated herein by reference in their entirety.

### FIELD OF THE INVENTION

The present invention relates to systems and methods utilizing one or more implantable devices for delivering electrical energy in the body. Specifically outlined is the use of a magnetostrictive electroactive (ME) implantable device for generating electrical energy via an externally applied magnetic field. The energy generated may be stored and/or used for continuous or temporary therapeutic intervention, such as stimulating bone growth.

### BACKGROUND OF THE INVENTION

Bone growth is naturally stimulated by mechanical stress; bone growth occurs to strengthen the parts that support the greatest load. It was discovered in the 1950s that bones are piezoelectric, that is, the application of a stress on bones generates a voltage across the stressed region. This led to the realization that bone growth after fracture can be enhanced by application of an electric field. Electrical current densities produced by such fields are typically in the range of 10 to 20 μA; 5 μA has been found to be ineffective and currents greater than 20 μA may cause bone death.

Electric field therapy can be applied either non-invasively from outside the body, or invasively with a surgically-implanted primary storage cell (battery) and electrodes (usually platinum) disposed across the fracture site.

Non-invasive electro-therapy for bone growth has been used more often for fractures of the appendicular skeleton. This method is usually referred to as capacitive coupling because the external electrodes, positioned across the fracture region, act as capacitor plates producing an electric field,

E=V/d=Q/(κ∈₀A), (1)

at the fracture site. Here, V is the voltage across the electrode plates, Q is the electric charge on the plates, κ is the relative permittivity, ∈₀=8.85×10⁻¹², is the dielectric constant, and A is the electrode plate area. Eq. 1 shows that the large separation of the electrodes used in non-invasive electrotherapy demands that a larger voltage be applied to achieve the same electric field at the fracture site. Capacitive coupling generally employs an externally worn 9 V battery, which must be changed almost daily. Non-invasive treatment can also be done with pulsed electromagnetic waves. Low-level pulsed electromagnetic fields (less than 100 Hz magnetic field of 0.01 to 0.1 T (100 to 1000 Oe) are found to be effective, as well as 1-10 Hz electromagnetic waves that typically draw 25-50 μA from the power source.

For invasive treatment of fractures that are non-healing or slow to heal, a spiral of wire that constitutes an electrode is surgically implanted inside the bone near the fracture point. This electrode is connected to a battery pack, which is implanted in nearby soft tissue. Another electrode extends from the battery pack and is attached to the bone surface. This is referred to as direct current (DC) stimulation. In this case the spacing, d, between the two electrodes is small compared to non-invasive procedures so that smaller voltages are sufficient to achieve the required electric field strength at the fracture site. Thus, the implanted battery can last longer than the 9 V external battery that is used in capacitive coupling. If it is assumed that the spacing of implanted electrodes is of order 5 to 10 mm, whereas the external electrodes are typically 100 to 200 mm apart, then Eq. 1 indicates that an implanted primary cell of the same capacity as a 9 V battery would last only 10 or 20 days. Electric field therapy is typically applied continuously for a period of up to 6 to 9 months. Therefore implanted primary cells must store a large amount of energy. While invasive bone stimulation is highly focused, the disadvantage is that a surgical procedure is required to implant the electrodes and large storage cell.

The problems met in non-invasive electrotherapy include 1) inconvenience, and hence a higher rate of patient non-compliance, due to the external apparatus that is currently used, 2) to get an adequate electric field to the precise location of the fracture using electrodes that are farther from the fracture or where the capacitor plates have a larger area, requires that the electric field fills a larger volume, thus drawing more current and requiring more power, 3) the larger field volume exposes more body tissue to potentially harmful currents, and 4) to sustain continuous field application to the fracture requires that a large electrical apparatus, housing a larger primary cell that produces a greater voltage, be attached to the body near the fracture.

The problems with invasive electrotherapy include 1) the need for surgical procedure and anesthesia to implant the electrodes and primary cell, 2) the need to adjust the location of the implanted electrodes during this surgery so that the field is applied directly across the fracture, and 3) despite the lower power requirements due to the proximity of the electrodes to the fracture site, implantation requires a relatively large implanted battery to provide continuous therapy for 6 to 9 months in order to avoid more frequent surgeries to replace the battery.

There is thus an ongoing need for improved systems and methods for delivering electrical energy in the body, for the purpose of promoting bone growth and other applications as well.

### SUMMARY OF THE INVENTION

An energy delivery device for use in various embodiments of the present invention is based on a class of magnetostrictive electroactive (ME) magnetic field components that produce a voltage when exposed to a changing magnetic field. The ME element is preferably a layered structure (e.g., sandwich) of magnetostrictive material bonded to an electroactive material. An external magnetic field causes a magnetization change in the magnetostrictive layer(s), which respond(s) with a magnetoelastic stress. Part of the stress is transferred to the electroactive layer that responds by producing a voltage given by Vᵢ=gᵢⱼσⱼLᵢ. Here, Lᵢ is the distance between the electrodes across which the voltage Vᵢ is measured, σⱼ is the stress transferred to the electroactive component, and gᵢⱼ is the stress-voltage coupling coefficient. The voltage is greatest when the direction i=j. However, in different applications the principal stress and induced voltage may lie in orthogonal directions (e.g., 1-3 operation), or the principal stress and voltage may act along different axes (e.g., 1-5 operation).

The ME device of the present invention is comprised of materials and dimensions designed preferably to produce a voltage and current that match the impedance of the load to be driven. The ME device is coupled to an electronic circuit that converts the AC output of the ME device to either AC or DC power for immediate use or for storage in a storage device (e.g., rechargeable battery or capacitor).

This new type of energy delivering device can be simpler, lighter and/or more compact than those requiring a permanent magnet as a field source. For example, suitable applications may include wireless monitoring applications, wherein wireless monitoring is meant to include self powered sensing of local conditions and processing of the sensor output and self powered wireless communication to a central data processing point. Other suitable applications include wireless transfer of electrical power over a small distance to a location inaccessible via electrical leads or not convenient for battery replacement. More specifically, these applications may include supplying power for:
wireless health monitoring or condition based therapy;
supplementing power or recharging batteries without physically accessing them;
elimination of wiring of electrical devices remote from a power source; and
powering of devices implanted in a living body (or to another inaccessible location) for purposes of sensing, transmitting, or actuating (e.g., motors, pumps, switches, valves, electrodes), as well as for accomplishing therapy or other functions that require a voltage and/or current.

In various embodiments, the invention addresses the above and other needs. It provides means for, e.g., acutely stimulating a nerve, spinal nerve, organ, soft tissue, incision site or similar, via a miniature implantable stimulator(s) that can be implanted via a minimal surgical procedure and powered by an external time-varying magnetic field.

The device of the present invention is a means of delivering power wirelessly from outside the body to inside the body for any of various therapies or health monitoring. The means of wireless power delivery consists of a magnetostrictive-electroactive (ME) magnetic-field element as the main component of a small implantable device that will receive a changing magnetic field from an alternating magnetic field source external to the body. This field source may be a hand-held device such as a small coil antenna or another ME device configured as a transmitter of alternating magnetic field. The external field source may be affixed to the wearer's skin, clothing or accessories. Alternatively, the external field may be generated by a source positionable on a chair, desk, car seat or table that the recipient frequents. The AC magnetic field excites the implanted ME device, which generates a voltage and current that can be used to provide therapeutic relief, e.g., by stimulating a nerve, bone or other tissue. The therapeutic system can be used to minimize tissue damage, reduce tumor size and burden, or stimulate bone or cartilage growth in the appropriate space. An externally applied AC magnetic field is a more efficient means of transmitting power than an AC electric field because of the greater attenuation of electric fields by body fluids.

The proposed system includes an external source of alternating magnetic field close to the recipient or in a wearable device that is made up of a power source (e.g., line power, battery, rechargeable battery or energy harvester) and electronics to control the signal generated by the wearable antenna (coil). The antenna is connected to the wearable device with a wire and affixed to the skin or cloth in the area of interest. In various embodiments, the antenna may perform two functions: 1) the antenna transmits data to communicate with the implanted devices; and 2) the antenna transmits a signal that is converted by the implanted ME receiver and/or other device into useful power for the entire implanted device.

The implanted ME device provides a very small implant that may be on a millimeter (mm) scale in size and can be driven by an external magnetic field applied only on an as needed basis. For example, patients with migraine headaches can be treated with therapeutic electricity applied to the occipital nerve. The patient does not however express this headache chronically, rather the headache appears only on an intermittently acute basis. Therefore applying the magnetic field during the earliest (prodromal) period of the headache can prevent conversion to a migraine headache. This would be one therapeutic embodiment of the technology.

Stimulation and control parameters of the implanted device may be preferably adjusted to levels that are safe and efficacious with minimal patient discomfort. Different stimulation parameters generally have different effects on neural tissue, and parameters are thus chosen to target specific neural populations and to exclude others. For example, large diameter nerve fibers (e.g., A-.alpha. and/or A-.beta. fibers) respond to relatively lower current density stimulation compared with small diameter nerve fibers (e.g., A-.delta. and/or C fibers). Stimulation patterns for non-neural therapy (tumor beds and incision sites) are delivered at the range of therapeutic efficacy.

The stimulator used in accordance with various embodiments of the present invention may possess one or more of the following properties: at least one implanted ME device to convert the externally applied magnetic field to electrical power for applying stimulating current to surrounding tissue and optionally acting as a sensor for determination of therapeutic efficacy and time constants related to the flow of current; electronic and/or mechanical components encapsulated in a hermetic package made from biocompatible material(s); an external coil that generates an AC magnetic field to deliver energy and/or information to the implanted ME wirelessly; a means for receiving and/or transmitting signals via telemetry; means for receiving and/or storing electrical power within the stimulator; and a form factor making the stimulator implantable via a minimal surgical procedure.

A stimulator may operate independently, or in a coordinated manner with other implanted devices, or with external devices. In addition, the device may incorporate means for sensing pain, which it may then use to control stimulation parameters in a closed loop manner. According to one embodiment of the invention, the sensing and stimulating means are incorporated into a single device. According to another embodiment of the invention, a sensing means communicates sensed information to at least one device with stimulating means.

Thus, in one embodiment the present invention provides a therapy for chronic pain that utilizes one or more miniature ME's as neurostimulators and is minimally invasive. The simple implant procedure results in minimal surgical time and possible error, with associated advantages over known treatments in terms of reduced expense, reduced operating time, single implant surgery, and therapy provided on an as needed basis. Other advantages, inter alia, of the present invention include the system's monitoring and programming capabilities, the power source, storage, and transfer mechanisms, the activation of the device by the patient or clinician, the system's open and closed-loop capabilities, the closed-loop capabilities being coupled with sensing a need for and/or response to treatment, coordinated use of one or more stimulators, and the small size of the stimulator.

In accordance with one embodiment of the invention, an apparatus is provided comprising:
an external source for transmitting a changing magnetic field into a human or other animal body; and
an implantable magnetostrictive-electroactive (ME) device that converts the changing magnetic field to generate electrical energy for use in the body.

In one embodiment, the apparatus of includes:

an implantable storage device coupled to the ME device for storing the electrical energy.

In another embodiment:

the ME device generates AC electrical energy and the apparatus includes a circuit that converts the AC electrical energy to DC electrical energy for immediate use and/or storage in the storage device.

In another embodiment:

the external source provides an adjustable magnetic field to adapt the electrical energy for a desired therapy.

In another embodiment:

the implanted ME device includes a circuit for conditioning the electrical energy for use in the body.

In another embodiment:

the implanted ME device includes a circuit for conditioning the electrical energy.

In another embodiment:

the external source comprises a coil driven by an AC current.

In another embodiment:

the external source comprises an applicator including one or more coils for positioning on the body.

In another embodiment:

the source comprises a coil surrounding a magnetic core.

In another embodiment:

the external source comprises a ME transmitter.

In another embodiment:

the storage device comprises a rechargeable cell or capacitor.

In another embodiment:

the apparatus includes a stabilizing rod, clamp or plate positionable within or in contact with a bone and comprising an electrode.

In another embodiment:

the apparatus including electrodes driven by the generated or stored electrical energy to produce an electric field in the body.

In one embodiment:

the electrical energy comprises an electric field adapted to promote bone growth.

In one embodiment:

the apparatus includes a pellet insertable in the body via a cannula or catheter, and the ME device is carried by the pellet.

In one embodiment

the pellet includes a circuit to process the generated electrical energy for storage in the storage device.

In one embodiment:

the apparatus includes a plurality of such pellets, each carrying an ME device.

In one embodiment:

the pellets have different resonant frequencies.

In one embodiment:

the pellet includes two electrodes on an outside surface of the pellet.

In accordance with one embodiment of the invention, a method is provided comprising:
providing a magnetostrictive-electroactive (ME) device and an electrical storage device inside a human or other animal body;
transmitting a changing magnetic field into the body;
the ME device converting the changing magnetic field to generate electrical energy; and
using the generated electrical energy for therapy in the body and/or storing the electrical energy in the storage device.

In one embodiment:

the generated and/or stored energy is used to produce an electric field in the body.

In another embodiment:

the generated and/or stored energy is used for electrical stimulation of bone growth.

In another embodiment:

the ME device generates AC electrical energy; and

converting the AC electrical energy to DC electrical energy for storage in the storage device.

In another embodiment, the method includes:

providing electrodes in the body and driving the electrodes with the generated and/or stored energy.

In another embodiment, the method includes:

providing one or more of the electrodes in or in contact with bone to provide electrical stimulation of bone growth.

In another embodiment, the method includes:

implanting one electrode in a bone and providing another electrode on an outer surface of a bone.

In another embodiment, the method includes:

the electrodes are positioned across a bone fracture.

In another embodiment, the method includes:

a spacing between the electrodes is in a range from 0.2 mm to 20 mm.

In another embodiment, the method includes:

generating an electrical current density in a range of 10 μA to 20 mA.

In another embodiment, the method includes:

generating an electrical current density in a range of 10 μA to 100 μA.

In another embodiment, the method includes:

using the generated and/or stored energy to deliver an electric field at bone fracture site.

In another embodiment, the method includes:

providing a magnetic field peak strength at the fracture site in a range of 10 A/m to 2 kA/m.

In another embodiment the method includes:

providing a magnetic field peak strength at the fracture site in a range of 80 A/m to 2 kA/m.

In another embodiment, the method includes:

the magnetic field having a frequency in a range of 30 kHz to 500 kHz.

In another embodiment, the method includes:

the storage device having a capacity on the order of kilowatt-hours.

In another embodiment, the method includes:

the storage device having a capacity of 1 milliWatt-hours to 20 kiloWatt hours.

In another embodiment, the method includes:

the storage device having a capacity of 100 Watt-hours to 20 kiloWatt hours.

In another embodiment, the method includes:

recharging the storage device by storing the generated energy.

In another embodiment, the method includes:

the storage device can be fully recharged in 30 minutes or less.

In another embodiment, the method includes:

delivering the generated and/or stored energy at transfer rate of 10 milliWatts to 1 Watt.

In another embodiment, the method includes:

delivering the generated and/or stored energy as a high voltage, low current signal for promotion of bone growth.

In another embodiment, the method includes:

the storage device has a capacity to deliver electrical energy for a given medical therapy for 10 days or less.

In another embodiment, the method includes:

providing an external source of the changing magnetic field on an outer surface of the body.

In another embodiment, the method includes:

the source comprising one or more coils wrapped around a limb of the body.

In another embodiment, the method includes:

the source comprising one or more coils placed against the skin.

These and other advantages of the present invention will be further understood by referring to the following detailed description and accompanying drawings.

### BRIEF DESCRIPTION OF THE DRAWINGS

FIG. 1 is a schematic diagram of one embodiment of magnetostrictive-electroactive (ME) element useful in the present invention;

FIG. 2 is a schematic diagram of a circuit for converting and changing the electrical output of an ME element to a DC electrical signal, conditioning, storing and providing the resulting electrical energy to a load;

FIG. 3 is a graph of ME voltage output versus field at 20 Hz for one embodiment of the invention;

FIG. 4 is a schematic diagram of a system of wireless power transmission for implanted devices; an external power source energizes a flat “patch” coil antenna that emits a magnetic-rich electromagnetic wave; an implanted magnetostrictive-electroactive (ME) sensor/transducer receives and converts the AC magnetic field to an AC voltage that is processed to produce the power needed for the a particular application;

FIG. 5 is a schematic diagram of another embodiment of an external changing magnetic field generated by a coil which is transmitted through tissue to an embedded ME device;

FIG. 6 is a graph of ME power versus electrical load at one (1) Oe and 29 kHz according to another embodiment of the invention;

FIG. 7 is a block schematic diagram of a magnetostrictive-electroactive device constructed in accordance with one embodiment of the invention;

FIG. 8 is a schematic diagram of an ME device (ME-33) showing a poled g₃₃ electroactive element with magnetostrictive outer layers bonded to the electroactive component and end electrodes providing for g₃₃ operation;

FIG. 9 is a schematic diagram of an alternative g₃₁ ME device;

FIG. 10 is a graph showing ME voltage output (mV) vs. magnetic field (mOe) in one ME-33 sensor with a sensitivity of 0.06 V/Oe;

FIG. 11 is a graph showing a decrease in magnetic field-per-Ampére (Oe/A) with distance b along a direction normal to a flat coil (cm);

FIG. 12 is a schematic illustration of one embodiment of an ME power receiver for use in stimulating bone growth; the opposing electrodes protruding from the large faces can be replaced by thin or thick film electrodes coplanar with the ME element;

FIGS. 13-14 are schematic illustrations of two types of coils that can be used to produce an AC magnetic field at the ME receiver site near the bone fracture in order to wirelessly generate the power needed to stimulate bone growth; the magnetic field direction in each case is suggested by the curved lines;

FIG. 15 is a schematic illustration of a bone fracture site in a patient's leg, wherein a coil electrode is positioned in the bone and another electrode is positioned on the outer surface of the bone, showing one embodiment of an implanted ME receiver and an external charger for simulating bone growth;

FIG. 16 is an alternative embodiment, similar to FIG. 15, but utilizing a stabilizing rod, embedded in the bone at the fracture site, as an electrode;

FIG. 17 shows outer and cross-sectional views of a titanium screw, of the type used to stabilize the bone during healing of a fracture, but here provided with an embedded ME receiver and provided in two parts, separated by insulation, which can act as two electrodes;

FIG. 18 is similar to FIG. 17 but takes the form of a bolt, rather than a screw, and can be used to connect two plates against a severe fracture; and

FIG. 19a-19c are schematic illustrations of another embodiment of the invention wherein ME pellets are inserted via a catheter (FIG. 19a) to the bone fracture site, the pellets are periodically recharged (FIG. 19b) by application of an external charger, and the patient enjoys continuous electrical therapy for bone growth over a period of one week without having to wear or carry the charger.

### DETAILED DESCRIPTION

FIG. 1 is a schematic illustration of one embodiment of an ME configuration 50 for use in the present invention. In this embodiment, a central layer of an electroactive (e.g., ceramic, polymer or single crystal piezoelectric; or a relaxor ferroelectric) material having a polarization vector P is shown, sandwiched between two layers 54, 56 of magnetostrictive material (e.g., of a soft ferromagnetic material having a non-zero magnetostriction) on opposing faces of central layer 52. Each magnetostrictive layer has a magnetization vector M which is caused to rotate in the plane of the magnetostrictive layer by an applied field H. A pair of electrodes 58 are disposed at opposite ends of the piezoelectric, the axis between the electrodes being parallel to the plane in which the magnetization vectors rotate. The voltage V generated in the piezoelectric, resulting from the magnetoelastic stress generated in the magnetic layers and transferred to the piezoelectric, can be measured in a circuit coupled to the electrodes attached to the piezoelectric layers.

The materials and configuration of the ME element may vary depending upon the particular application. While it is generally desirable to use a magnetic material with large magnetostriction for the magnetic layer(s), it is generally more important (for optimum power delivery) that the magnetostrictive material have a large product of a magnetostrictive stress and stiffness modulus (see “Novel Sensors Based on Magnetostrictive/Piezoelectric Lamination,” J. K. Huang, D. Bono and R. C. O'Handley, Sensors and Actuators 2006). This insures that the magnetic layer(s) more effectively transfer stress to the piezoelectric material. For example, while FeCo(Hyperco) shows a relatively large magnetostriction (approaching 100 ppm) and is extremely stiff, the product of these parameters translates to a magnetostrictive stress of 12 MPa. A high-magnetostriction material such as Fe₂(Dy_{2/3}Tb_{1/3}) (known as Terfenol-D) on the other hand, is mechanically softer than FeCo but shows a much larger magnetostrictive strain and its magnetostrictive stress approaches 60 MPa.

It is also important (for optimum power generation) that the magnetostrictive stress changes by the largest possible amount under the influence of the changing field strength. For example, the magnetization vector of FeCo can be rotated in a field of a few tens of Oe (Oersteds) while the magnetization vector of Terfenol-D can be rotated in a field of several hundreds of Oe, provided in each case they are properly annealed and the aspect ratio of the material in the magnetizing direction is favorable.

The class of magnetostrictive materials that can be magnetized in the weakest fields consist of a variety of amorphous alloys based on iron (Fe) (optionally in combination with nickel (Ni)) and with glass formers such as boron (B) (optionally with silicon (Si)).

Electroactive materials, such as the commercially available piezoelectric lead-zirconate-titanates (PZT) have stress-voltage coefficients, g₁₃ and g₃₃, with values approximately equal to 10 and 24 mV/(Pa-m), respectively. Thus, a stress applied to the piezoelectric parallel to the direction across which the voltage is measured is more effective in generating a voltage than a stress transverse to this direction (out of the plane in which the vector is rotated by the field). Further, relaxor ferroelectrics have gᵢⱼ values that can be three to four times those of piezoelectrics. Also useful in these applications are piezo fibers or manufactured piezo fiber composites. They may have interdigitated electrodes with various spacings to produce electric fields along the piezo fibers or they may be electroded across the thickness of the fibers. Polymeric piezoelectric material(s) (e.g., poly-vinylidene-difluoride PVDF) may be advantageous in some applications.

There are a number of ways to increase the strength of the applied magnetic field entering the magnetostrictive layers so as to enhance the power generated. One way is to use flux concentrators (e.g., fan-shaped soft magnetic layers) placed in series with the ME element in the presence of the field.

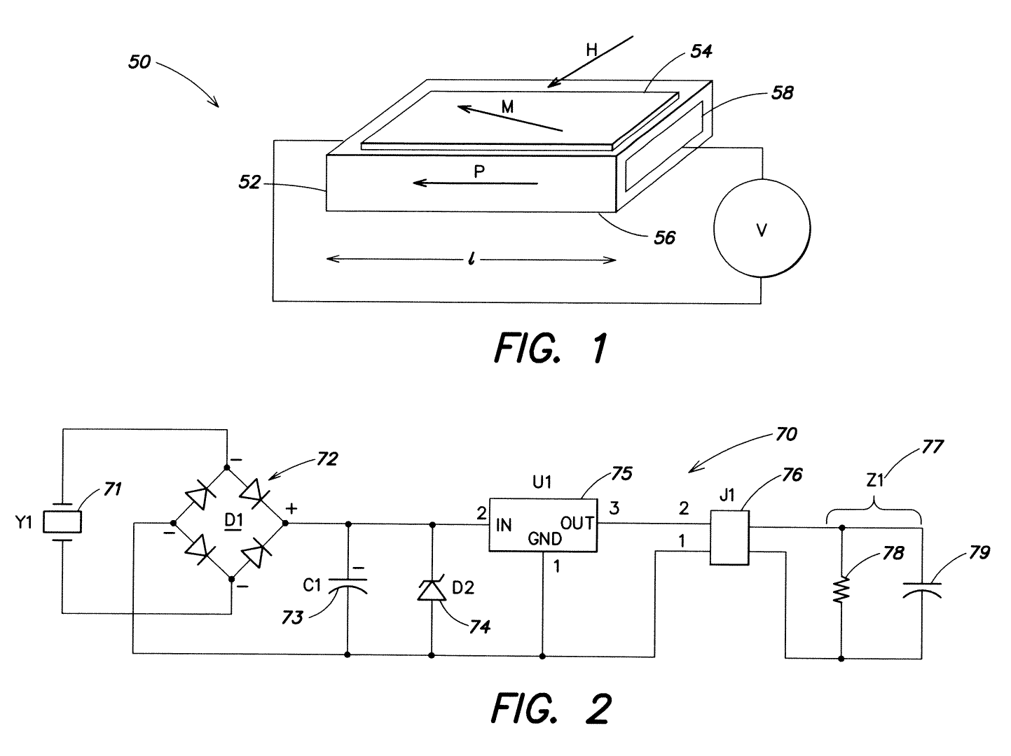

The electrical energy output of the ME device can be adapted for immediate use or storage by coupling the ME device to an electronic circuit. One such circuit 70 is shown in FIG. 2. On the left hand side, an ME element (Y1) 71 is shown. A diode bridge (D1) 72 is disposed in parallel across the ME output. The full wave diode bridge converts the AC electric charge on the ME element to a DC charge. Connected in parallel to the diode bridge is an energy storage capacitor (C1) 73 which stores the energy generated by the ME element as a voltage across it. Alternatively, the storage capacitor 73 can be replaced by a rechargeable battery. Parallel to the capacitor is a limiter zener diode (D2) 74 which prevents overcharging of the capacitor C1 beyond its breakdown voltage. Next provided in parallel to the capacitor and diode bridge is a voltage regulator (U1) 75. The voltage regulating circuit reduces the capacitor voltage to a useful level for a load. The voltage regulated output across (J1) 76 is applied to the load, here represented as a load impedance (Z1) 77, which typically includes resistive 78 and capacitive 79 elements, and which uses the generated electrical energy to do useful work.

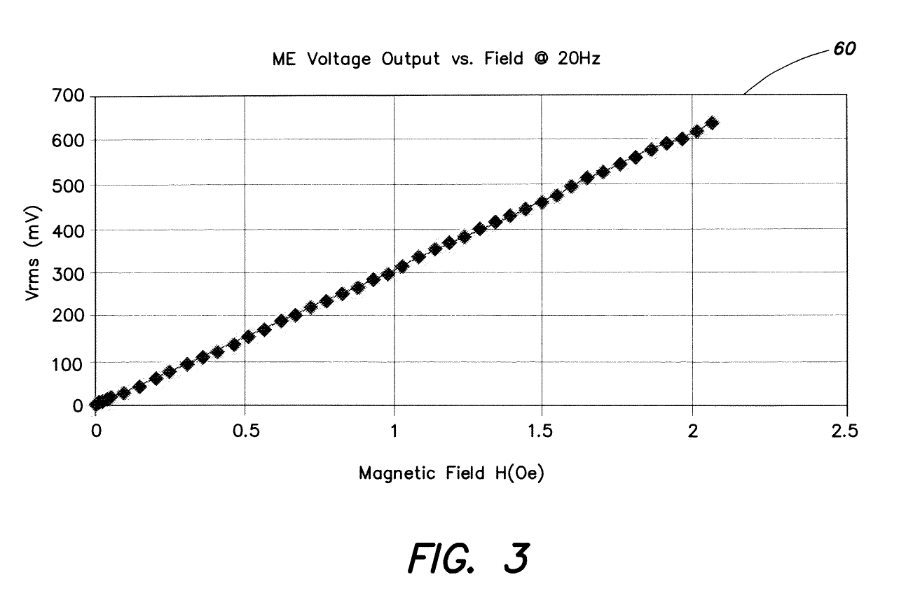

FIG. 3 is a graph 60 comparing the ME output voltage signal (RMS voltage in millivolts) versus magnetic field strength (H in telsa). In this embodiment, the changing external magnetic field is at a low frequency of 20 Hz. The ME voltage output linearly increases from 0 to 650 millivolts with increasing magnetic field strength from 0 to 20e. The substantially linear relationship between the ME voltage output and magnetic field strength is representative for low frequency applications (where the field changes are at low frequencies).

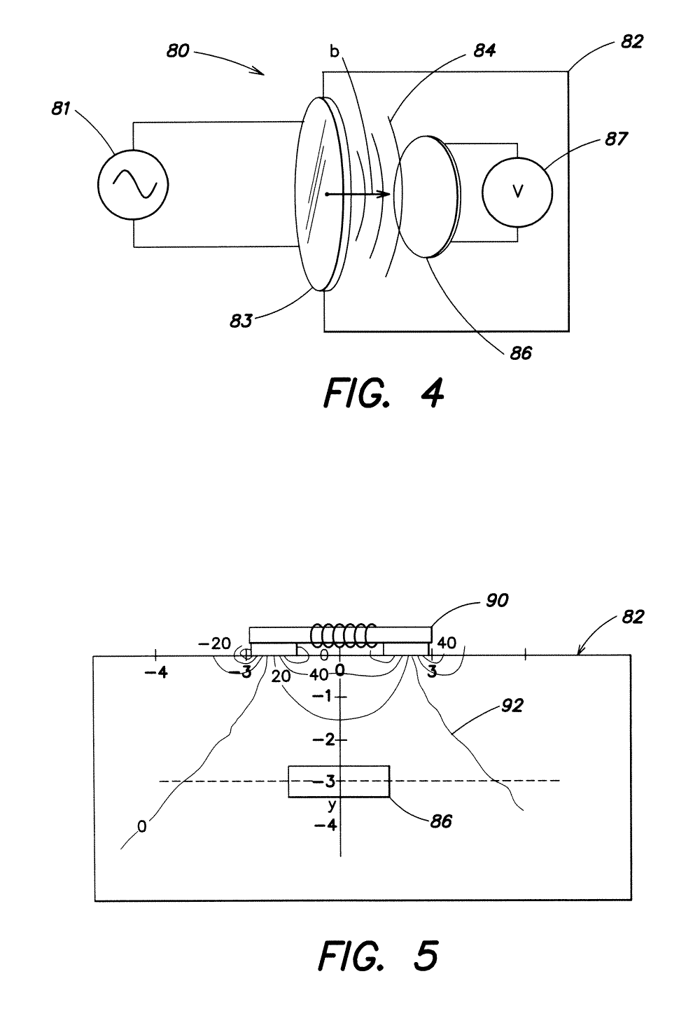

Alternatively, a higher frequency external field can be used to obtain a greater level of power from the ME (compared to the low frequency operation of FIG. 3). This is illustrated with the embodiment and resulting power output shown in FIGS. 4-5. FIG. 4 illustrates a system 80 for delivering power inside a living organism (or any inaccessible or difficult to access location) without requiring the use of electrical wiring between the source of the power and the target device and without requiring (or with diminished need for) batteries. FIG. 4 shows an external power source (e.g., battery) 81 which powers an external loop antenna 83 generating an alternating magnetic field 84 outside of the body. The magnetic field 84 generated by this loop antenna is transmitted (in a direction b normal to the phane of the coil 83) through the skin and other tissue 82 to an embedded ME element 86 producing a resulting output voltage V (87). The power transmission here is achieved not by a high frequency microwave, RF or other electromagnetic wave, but rather by means of a relatively low frequency, benign, alternating magnetic field. Microwaves and other electromagnetic waves having a wavelength comparable to or less than the distance between the source and receiver, are rapidly attenuated by water or metals, and thus would not be suitable in this application. Instead, the loop antenna produces low-frequency, magnetic-rich waves which are left essentially unattenuated by tissue (assuming no intervening magnetic material), and which do not have problems with tissue heating that accompanies microwaves. Alternatively, instead of a loop antenna (with no core) the external source could be a core-filled coil antenna such as a solenoid coil with core 90 as shown in FIG. 5, wherein the core may significantly enhance the field 92 in the body.

The field generated by a loop, solenoid or core-filled coil antenna is richer in magnetic field strength than electric field strength within a range comparable to the wavelength of the radiation. The wavelength is given by the equation λ=c/f, where c is the speed of light in the medium, and f is the frequency of the radiation. At 1 MHZ (megahertz) in air, λ equals 300 m (meters); at 100 MHZ, λ equals 3 m. Thus, there is a wide range of frequencies over which to transmit a magnetic-field rich electromagnetic wave without significant attenuation.

The implanted magnetostrictive-electroactive (ME) sensor/transducer 86 receives and converts the AC magnetic field 84/92 to an AC voltage that can be processed to produce power needed for a particular application. For example, this apparatus can be used in powering internal pumps, sensors, valves and transponders in human and animals. More generally, it can be used to power devices which monitor health, organ function or medication needs, and for performing active functions such as pumping, valving, stimulation of cell growth or accelerated drug or radiation treatment locally. The described means of delivering power inside a living organism can be achieved without the use of electrical wires inbetween the source of the power and the target receiver (ME device) and without the need, or diminished need, for batteries.

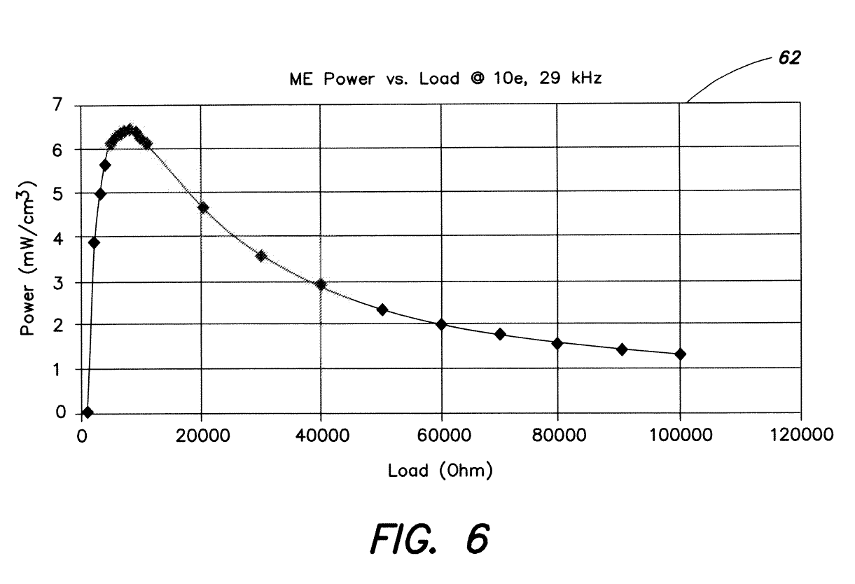

The wireless power transmitted can be optimized, for example, if resonance is achieved at each stage of transmission. Thus the external power source and the transmit antenna may be in resonance. The ME device may also be in resonance with the magnetic field it responds to, and the ME device may also be in resonance with the part of the circuit that receives the signal from the ME device. By careful design and material selection, it is possible for all three resonances to closely coincide. FIG. 6 illustrates one example of a ME power output (mW/cm³) versus load (ohm) for one such resonant system operating at a frequency of 29 kHz and a field of one (1) Oe.

There will now be described in more detail alternative device configurations and materials which may be useful in various embodiments of the present invention.

FIG. 7 is a block schematic diagram of one embodiment of a magnetostrictive electroactive element useful in the practice of the invention. The device 400 comprises a magnetic layer 402 that is bonded to a piezoelectric layer 404 by a suitable non-conductive means, such as non-conductive epoxy glue. Although only one magnetic layer 402 is shown bonded to a single piezoelectric layer 404, those skilled in the art would understand that two or more magnetic layers can be used. The magnetization vector 415 (M) of the magnetic material 402 rotates in the plane 416 of the magnetic layer 402 when an external magnetic field (H) is applied as shown by the arrow 414. The rotation of the magnetization vector M causes a stress in the magnetostrictive layer 402 which is, in turn, applied to the piezoelectric layer 404 to which the magnetic layer 402 is bonded. In this design the direction of magnetization, M, rotates in the preferred plane of magnetization, changing direction from being parallel to perpendicular (or vice versa) to a line joining the electrodes. This maximizes the stress change transferred to the electroactive element.

The stress-induced voltage in the piezoelectric material 404 is measured across a pair of electrodes 406 and 407 of which only electrode 406 is visible in FIG. 7 (electrode 407 being positioned on the device end opposite to electrode 406). The magnitude of the voltage developed across electrodes 406 and 407 is a function of the magnetic field strength for H<Hₐ, the anisotropy field (at which M is parallel to the applied field) and can be utilized to power a device 410 that is connected to electrodes 406 and 407 by conductors 412 and 408, respectively.

The ME device is constructed so that stress-induced voltage is measured in a direction that is parallel to the plane 416 in which the magnetization rotates. The stress is generated in the magnetic material 402, which responds to an external magnetic field 414 (H) with a magnetoelastic stress, σ_{mag}, that has a value in the approximate range of 10 to 60 MPa. Because the magnetic material 402 is bonded to a piezoelectric layer 404, the layer 404 responds to the magnetostrictive stress with a voltage proportional to the stress, σ_{mag}, transmitted to it. Piezoelectric materials respond to a stress with a voltage, V, that is a function of the applied stress, a voltage-stress constant, gᵢⱼ, and the distance, l between the electrodes. In particular,

δV=gᵢⱼ^{piezo}fδσ_{mag}l

Here δσ_{mag} is the change in magnetic stress that is generated in the magnetic material by the field-induced change in its magnetization direction. A fraction, f, of this stress is transferred to the electroactive element. δV is the resulting stress-induced change in voltage across the electrodes on the electroactive element.

If the voltage is measured in a direction orthogonal to the direction in which the stress changes, then gᵢⱼ=g₁₃. As mentioned previously, typically piezoelectric values for g₁₃ are 10 millivolt/(meter-Pa). However, if the voltage is measured in a direction parallel to the principal direction in which the stress changes, then gᵢⱼ=g₃₃. Thus, the sensor operates in a g₃₃ or d₃₃ mode. For a typical piezoelectric material g₃₃=24 millivolt/(meter-Pa)=0.024 volt-meter/Newton. In this case, a stress of 1 MPa generates an electric field of 24 kilovolt/meter. This field generates a voltage of 240 V across a 1 cm (l=0.01 m) wide piezoelectric layer.

The stress generated by the magnetic material 402 depends on the extent of rotation of its magnetization, a 90 degree rotation producing the full magnetoelastic stress. The extent of the rotation, in turn, depends of the angle between the magnetization vector 415 and the applied magnetic field direction 414 and also depends on the strength of the magnetic field and on the strength of the magnetic anisotropy (magnetocrystalline, shape and stress-induced) in the magnetic layer. The fraction, f, of the magnetostrictive stress, σ_{mag}, transferred from magnetic to the piezoelectric layer depends on the (stiffness×thickness) product of the magnetic material, the effective mechanical impedance of the bond between the magnetic and electric elements (proportional to its stiffness/thickness), and the inverse of the (stiffness×thickness) of the piezoelectric layer.

A quality factor may be defined from the above equation to indicate the sensitivity of the device, that is, the voltage output per unit magnetic field, H (Volts-m/A):

$$\frac{\partial V}{\partial H} = {g}_{33}^{piezo} f ( \frac{\partial {σ}_{mag}}{\partial H} ) l$$

The characteristics of a preferred magnetostrictive material are a large internal magnetic stress change as the magnetization direction is changed. This stress is governed by the magnetoelastic coupling coefficient, B₁, which, in an unconstrained sample, produces the magnetostrictive strain or magnetostriction, λ, proportional to B₁ and inversely proportional to the elastic modulus of the material. In general, the magnetic material is also mechanically robust, relatively stable (not prone to corrosion or decomposition), and receptive to adhesives. In addition, if the magnetic material is electrically non-conducting, it can be bonded to the electroactive element with the thinnest non-conducting adhesive layer that provides the needed strength without danger of shorting out the stress-induced voltage developed across the electroactive element. For ME devices in which the voltage is measured across electrodes that are not the same as the magnetostrictive layers, care must be taken that the magnetostrictive layers not short out the voltage between the measuring electrodes. This can be accomplished by using a non-conducting adhesive to insulate the magnetostrictive layer(s) from the electroactive element(s).

Many known magnetostrictive materials can be used for the magnetic layer 402. These include various magnetic alloys, such as amorphous-FeBSi or Fe—Co—B—Si alloys, as well as polycrystalline nickel, iron-nickel alloys, or iron-cobalt alloys such as Fe₅₀Co₅₀ (Hyperco). For example, amorphous iron and/or nickel boron-silicon alloys of the form FeₓB_{y}Si_{1-x-y}, where 70<x<86 at %, 2<y<20, and 0<z=1−x−y<8 at % are suitable for use with the invention with a preferred composition near Fe₇₈ B₂₀Si₂. Also suitable are alloys of the form FeₓCo_{y}B_{z}Si_{1-x-y-z} where 70<x+y<86 at % and y is between 1 and 46 at %, 2<z<18, and 0<1−x−y−z<16 at %, with a preferred composition near Fe₆₈Co₁₀B₁₈Si₄. Iron-nickel alloys with Ni between 40 and 70 at % with a preferred composition near 50% Ni can be used. Similarly, iron-cobalt alloys with Co between 30 and 80% and a preferred composition near 55% Co (such as Fe₅₀Co₅₀.) are also suitable.

Another magnetostrictive material that is also suitable for use with the invention is Terfenol-D® (TbₓDy₁₋ₓFe_{y}), an alloy of rare earth elements Dysprosium and Terbium with the transition metal iron, manufactured by ETREMA Products, Inc., 2500N. Loop Drive, Ames, Iowa 50010, among others. Terfenol-D® can generate a maximum stress on the order of 60 MPa for a 90-degree rotation of its magnetization. Such a rotation can be accomplished by an external applied magnetic field on the order of 400 to 1000 Oersteds (Oe). Also useful are highly magnetostrictive alloys such as Galfenol®, Fe₁₋ₓGaₓ. (ETREMA Products). Softer magnetic materials, such as certain Fe-rich amorphous alloys mentioned above, may achieve full rotation of magnetization in fields of order 10 Oe, making them suitable for the magnetic layer in a sensor for sensing weaker fields. Finally, it is possible to use certain so-called nanocrystalline magnetic materials. In these polycrystalline materials, it is generally that case that the magnetization can be rotated as easily as it can be in amorphous materials. But nanocrystalline materials can be engineered to have larger magnetoelastic coupling coefficients than amorphous materials.

The preferred characteristics of a suitable electroactive layer for the sensor devices are primarily that they have a large stress-voltage coupling coefficient, g₃₃. In addition, they preferably are mechanically robust, receptive to adhesives, not degrade the metallic electrodes (this is most often easily achieved when the electrodes are made of noble metals, such as silver or gold). Generally, the electroactive material is chosen on the basis of having a value of gᵢⱼ greater than 10 mV/(Pa-m).

The electroactive layer can be a ceramic piezoelectric material such as lead zirconate titanate Pb(ZrₓTi₁₋ₓ)O₃, or variations thereof, aluminum nitride (AlN) or simply quartz, SiOₓ. In some applications a single crystal (as opposed to a ceramic or polycrystalline) piezoelectric material may be advantageous. Alternatively, a polymeric piezoelectric material such as polyvinylidene difluoride (PVDF) would be suitable for applications where the stress transferred from the magnetostrictive material is relatively weak. The softness of the polymer will allow it to be strained significantly under weaker applied stress to produce a useful polarization, or voltage across its electrodes. It is also advantageous in some applications to use another electroactive material, such as an electrostrictive material (for example, (Bi_{0.5}Na_{0.5})₁₋ₓBaₓZr_{y}Ti_{1-y}O₃) or a relaxor ferroelectric material (for example, Pb(Mg_{1/3}Nb_{2/3})₃). Collectively, the piezoelectric, ferroelectric, electrostrictive and relaxor ferroelectric layers are called “electroactive” layers.

Piezoelectric materials typically have g₃₃˜4×g₃₁ and g₃₃≈20 to 30 mV/(Pa-m) which is about 10×d₃₁. For PVDF, g₃₃≈100 mV/(Pa-m) and some relaxor ferroelectrics can have g₂₂≈60 mv/(Pa-m).

Model predictions and experimental results shown in Table 1 compares the parameters gᵢⱼ, in mV/m-Pa, the electrode spacing l in meters, the maximum stress per unit field (B₁/μₒHₐ) in Pa/T, and calculated field sensitivity in nV/nT and the observed field sensitivity, dV/dB. The values tabulated for a g₃₃ device using a relaxor ferroelectric are based on the data observed with a piezoelectric based sensor and using a ratio of g₃₃ for typical relaxors/piezoelectrics.

TABLE 1

| | | | max. | Sensitivity | |
|---|---|---|---|---|---|
| | gᵢⱼ | l | stress | Calc. | Obs. |
| Piezo/magnetic sensors: | | | | | |
| d₃₁ sensor | 11 | 10⁻³ | 10⁸ | 10⁴ | 280 |
| d₃₁ sensor | 11 | 10⁻³ | 10⁸ | 10⁴ | 1,200 |
| d₃₃ sensor | 24 | 10⁻² | 10⁹ | 2 × 10⁵ | 1.5 × 10⁴ |
| Relaxor/magnetic sensors: | | | | | |
| d₃₃ relaxor/mag sensor | 60 | 10⁻² | 10⁹ | 10⁶ | (10⁵) |

The calculated sensitivity in the table is defined with perfect stress coupling, namely a quality factor Q=1 in MKS units (V/Tesla) as

$$\frac{\partial V}{{μ}_{o} \partial H} \approx {g}_{33}^{piezo} l \frac{{B}_{1}}{{μ}_{o} {H}_{a}}$$

Here B₁ is the magneoelastic coupling coefficient, a material constant that generates the magnetic stress in the magnetostrictive material, σₘ, which was used in earlier equations.

Other useful sensor embodiments are disclosed in U.S. Ser. No. 10/730,355 filed 8 Dec. 2003 entitled “High Sensitivity, Magnetic Field Sensor and Method of Manufacture,” by J. Huang, et al., the subject matter of which is incorporated by reference herein in its entirety.

### Medical Background

Various medical applications for the energy transmission system of the present invention will now be described.

Chronic pain is a multidimensional phenomenon involving complex physiological and emotional interactions. For instance, one type of chronic pain, complex regional pain syndrome (CRPS)—which includes the disorder formerly referred to as reflex sympathetic dystrophy (RSD)—most often occurs after an injury, such as a bone fracture. The pain is considered “complex regional” since it is located in one region of the body (such as an arm or leg), yet can spread to additional areas. Since CRPS typically affects the sympathetic nervous system, which in turn affects all tissue levels (skin, bone, etc.), many symptoms may occur. Pain is the main symptom. Other symptoms vary, but can include loss of function, temperature changes, swelling, sensitivity to touch, and skin changes.

Another type of chronic pain, failed back surgery syndrome (FBSS), refers to patients who have undergone one or more surgical procedures and continue to experience pain. Included in this condition are recurring disc herniation, epidural scarring, and injured nerve roots.

Arachnoiditis, a disease that occurs when the membrane in direct contact with the spinal fluid becomes inflamed, causes chronic pain by pressing on the nerves. It is unclear what causes this condition.

Yet another cause of chronic pain is inflammation and degeneration of peripheral nerves, called neuropathy. This condition is a common complication of diabetes, affecting 60%-70% of diabetics. Pain in the lower limbs is a common symptom.

An estimated 10% of gynecological visits involve a complaint of chronic pelvic pain. In approximately one-third of patients with chronic pelvic pain, no identifiable cause is ever found, even with procedures as invasive as exploratory laparotomy. Such patients are treated symptomatically for their pain.

A multitude of other diseases and conditions cause chronic pain, including postherpetic neuralgia and fibromyalgia syndrome. Neurostimulation of spinal nerves, nerve roots, and the spinal cord has been demonstrated to provide symptomatic treatment in patients with intractable chronic pain.

Many other examples of chronic pain exist, as chronic pain may occur in any area of the body. For many sufferers, no cause is ever found. Thus, many types of chronic pain are treated symptomatically. For instance, many people suffer from chronic headaches/migraine and/or facial pain. As with other types of chronic pain, if the underlying cause is found, the cause may or may not be treatable. Alternatively, treatment may be only to relieve the pain.

Chronic pain, though the primary indication for neurostimulation, is not the only disease entity in the human body that can benefit from neuromodulation. Treatment of acute stroke, sleep apnea, cancer, migraines, bone and joint disease and various types of primary brain disorders such as depression, epilepsy and mood disorders would benefit greatly from neuromodulation.

The devices currently available for producing therapeutic stimulation have drawbacks. Many are large devices that must apply stimulation transcutaneously. For instance, transcutaneous electrical nerve stimulation (TENS) is used to modulate the stimulus transmissions by which pain is felt by applying low-voltage electrical stimulation to large peripheral nerve fibers via electrodes placed on the skin. TENS devices can produce significant discomfort and can only be used intermittently.

Other devices require that needle electrode(s) be inserted through the skin during stimulation sessions. These devices may only be used acutely, and may cause significant discomfort.

Implantable stimulation devices are available, but these currently require a significant surgical procedure for implantation. Surgically implanted stimulators, such as spinal cord stimulators, have different forms, but are usually comprised of an implantable control module to which is connected a series of leads that must be routed to nerve bundles in the spinal cord, to nerve roots and/or spinal nerves emanating from the spinal cord, or to peripheral nerves. The implantable devices are relatively large and expensive. In addition, they require significant surgical procedures for placement of electrodes, leads, and processing units. These devices may also require an external apparatus that needs to be strapped or otherwise affixed to the skin. Drawbacks, such as size (of internal and/or external components), discomfort, inconvenience, complex surgical procedures, and/or only acute or intermittent use has generally confined their use to patients with severe symptoms and the capacity to finance the surgery.

There are a number of theories regarding how stimulation therapies such as transcoutaneous electrical neuro-stimulation (TENS) machines and spinal cord stimulators may inhibit or relieve pain. The most common theory—gate theory or gate control theory—suggests that stimulation of fast conducting nerves that travel to the spinal cord produces signals that “beat” slower pain-carrying nerve signals and, therefore, override/prevent the message of pain from reaching the spinal cord. Thus, the stimulation closes the “gate” of entry to the spinal cord. It is believed that small diameter nerve fibers carry the relatively slower-traveling pain signals, while large diameter fibers carry signals of e.g., touch that travel more quickly to the brain.

Spinal cord stimulation (also called dorsal column stimulation) is best suited for back and lower extremity pain related to adhesive arachnoiditis, FBSS, causalgia, phantom limb and stump pain, and ischemic pain. Spinal cord stimulation is thought to relieve pain through the gate control theory described above. Thus, applying a direct physical or electrical stimulus to the larger diameter nerve fibers of the spinal cord should, in effect, block pain signals from traveling to the patient's brain. In 1967, Shealy and coworkers first utilized this concept, proposing to place stimulating electrodes over the dorsal columns of the spinal cord. (See Shealy C. N., Mortimer J. T., Reswick, J. B., “Electrical Inhibition of Pain by Stimulation of the Dorsal Column”, in Anesthesia and Analgesia, 1967, volume 46, pages 489-491.) Since then, improvements in hardware and patient selection have improved results with this procedure.

The gate control theory has always been controversial, as there are certain conditions such as hyperalgesia, which it does not fully explain. The relief of pain by electrical stimulation of a peripheral nerve, or even of the spinal cord, may be due to a frequency-related conduction block which acts on primary afferent branch points where dorsal column fibers and dorsal horn collaterals diverge. Spinal cord stimulation patients tend to show a preference for a minimum pulse repetition rate of 25 Hz.

Stimulation may also involve direct inhibition of an abnormally firing or damaged nerve. A damaged nerve may be sensitive to slight mechanical stimuli (motion) and/or noradrenaline (a chemical utilized by the sympathetic nervous system), which in turn results in abnormal firing of the nerve's pain fibers. It is theorized that stimulation relieves this pain by directly inhibiting the electrical firing occurring at the damaged nerve ends.

Stimulation is also thought to control pain by triggering the release of endorphins. Endorphins are considered to be the body's own pain-killing chemicals. By binding to opioid receptors in the brain, endorphins have a potent analgesic effect.

Recently, an alternative to 1) TENS, 2) percutaneous stimulation, and 3) bulky implantable stimulation assemblies has been introduced. Small, implantable stimulators have been introduced that can be injected into soft tissues through a cannula or needle. The most specific of these, the bion, can produce electrical energy through a tiny battery that does not require wires or leads to be active. The negative with this therapy however is that the recharge capacity of these products is very poor, and that they do not have the capacity to deliver therapy for prolonged periods of time. In addition, like all other neurostimulators, these products are designed for continual stimulation therapy. There are a wide variety of indications that are not treated by current neurostimulation device methods which require therapy only on an as needed basis. The therapy is ongoing, however it isn't continual throughout the day. Providing therapy in this manner will allow for the introduction of a product that can be miniscule in size and be driven by limited power without sacrificing long term viability of the device itself.

### ME-33 Embodiment of an Implantable ME Device

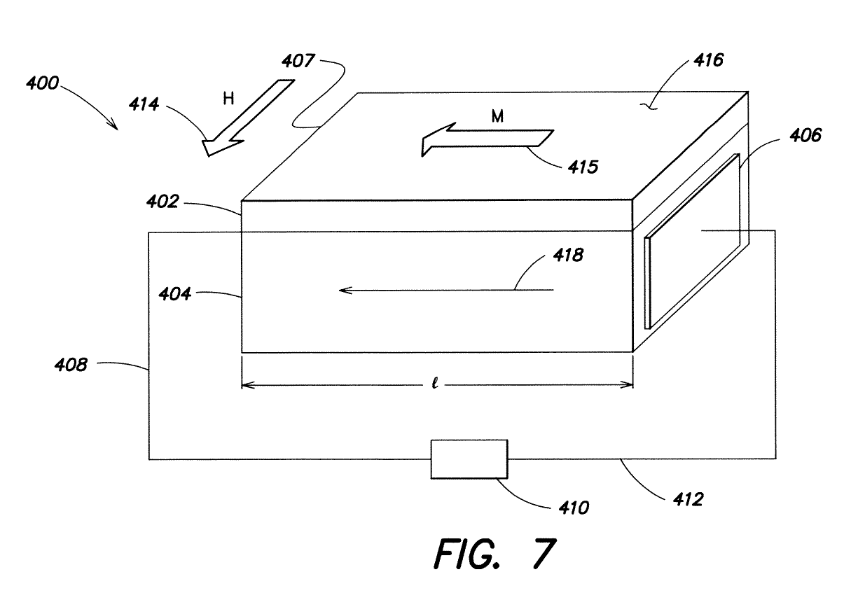

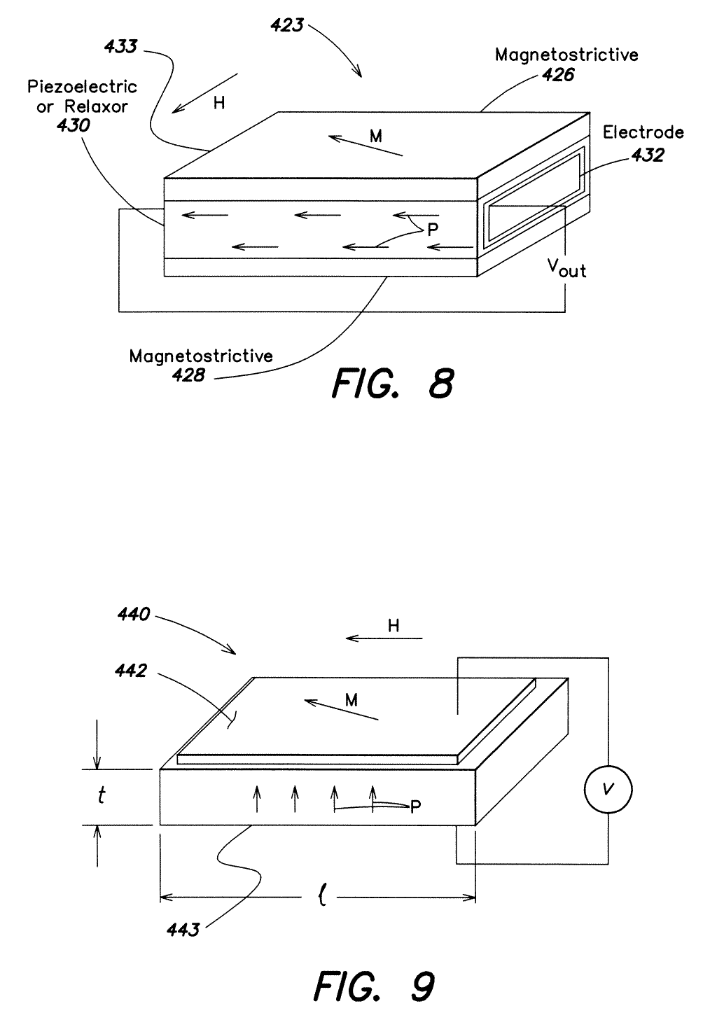

One embodiment of an implantable ME device is a high-sensitivity, magnetostrictive-electroactive magnetic field element, e.g., g₃₃ mode (ME-33) device developed by Ferro Solutions, Inc., Woburn, Mass., USA, depicted schematically in FIG. 8 as ME element 423. Other magnetostrictive-electroactive devices, e.g. a g₃₁ device 440 having opposing electrodes 442, 443 disposed on the top and bottom large faces of the device as shown in FIG. 9, may also be used with this system. The ME device of FIG. 8 converts magnetic field variations seen by the two outer magnetic layer(s) 426, 428 to a magnetostrictive stress, σ, some of which is transmitted to the central electroactive element 430 to which it is bonded. The stressed electroactive element develops a voltage Vout across its opposing end electrodes 432, 433. The coupling coefficient g₃₃ can have values ranging from about 0.015 V/m-Pa for ceramic PZT, to 0.1 V/m-Pa for single-crystal electroactive materials. Typically, g₃₃ is 3 or 4 times greater than g₃₁ used in all other reported, prior-art ME sensors. Magnetostrictive stresses can be as large as 60 MPa [for Terfenol-D, Fe₂(Tb,Dy)]. Thus, the ME-33 sensors have a theoretical limiting magneto-electric sensitivity of order 6×10⁷ V/(m-T), much greater than that of a similar g₃₁ device. ME-33 devices similar to that shown in FIG. 8 exhibit a linear response of 5 V/Oe with fields as small as 2 μT (20 mOe). One can further enhance the sensitivity of this device by applying an AC bias to the magnetic layer(s).

A magneto-electric sensitivity or quality factor, Qₘₑ (V/T) can be defined for ME devices. One can estimate the limiting values for Qₘₑ using known material parameters. One material combination is amorphous magnetic alloy and PVDF electroactive material. Other material combinations include either Terfenol-D or Fe—Co magnetostrictive layers with PZT electroactive materials. These devices typically produce 10s of V/Oe. One could also use Fe—Ga magnetostrictive material with any of the electroactives including single-crystal or electrostrictive materials. For a 1 cm long sensor (L≈10−2 m), the theoretical output voltage per unit field is:

Qₘₑ^{amorph-PVDF}≈2.1×10⁵(V/T)[Qₘₑ^{amorph-PVDF}=21(V/Oe)] (1)

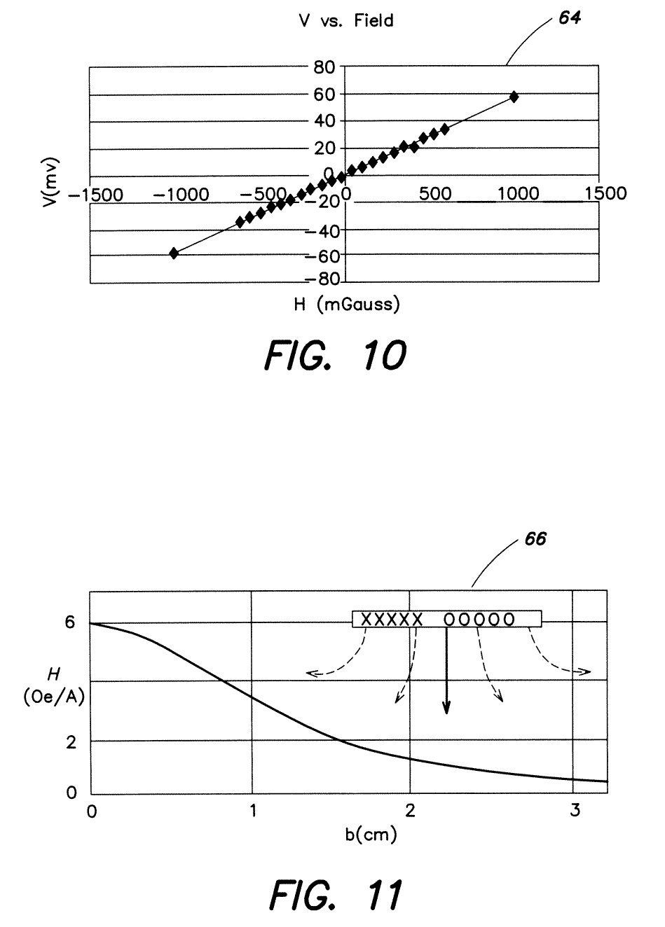

FIG. 10 is a graph showing ME voltage output (mV) vs. magnetic field (mOe) in one ME-33 sensor with a sensitivity of 0.06 V/Oe.

As illustrated in FIG. 4, a magnetic field 84 for driving the ME element (86 in FIG. 4, 423 in FIG. 9) (and thus making it function as a remote voltage generator) is provided by an external (of the body) power source (e.g., battery) 81 that is connected to a small external flat-profile coil antenna 83 positioned adjacent the patient's skin 82. Such an antenna generates a near field that is mainly magnetic in character. The voltage V (87) generated by the ME device 86/423 is available for storage (and later use) or immediate use for electrical stimulation in the body.

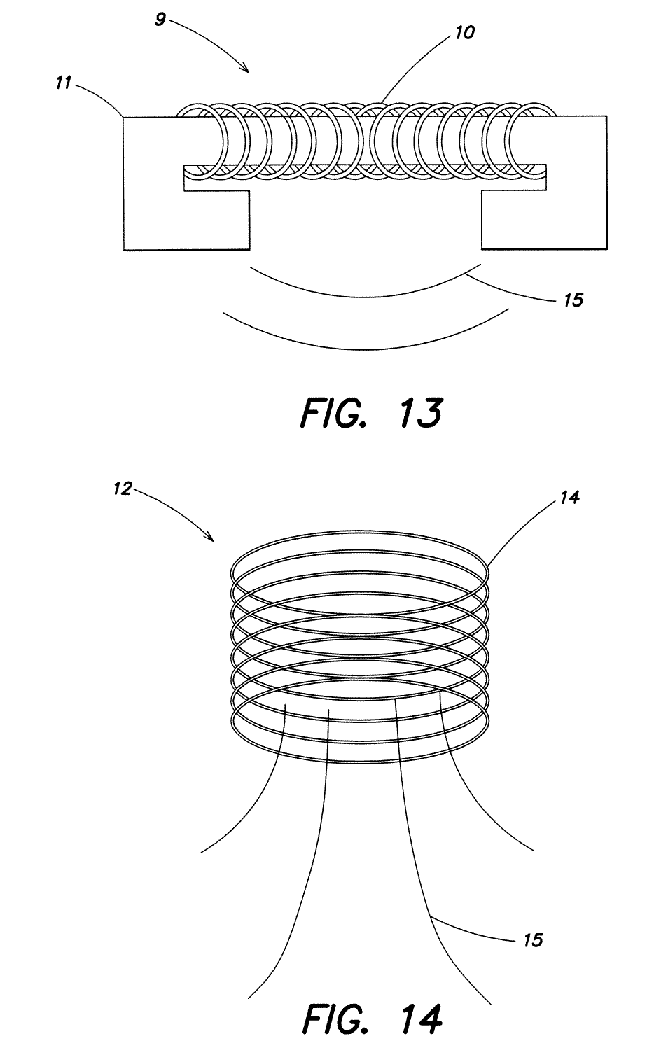

For example, consider a 3 cm-diameter, ten-turn coil (e.g., FIGS. 13-14) with each turn (made from wire or a flat foil or film) having a cross section of 0.05×1 mm; this coil would have a resistance of less than 1 Ohm. When driven by a 1 VDC signal (possibly battery-powered), it would draw a current of a few tens of Amperes producing a field on the order of 5-10 Oe, 1 or 2 cm beneath the coil. This is more than enough to produce a significant rotation in the magnetization of the ME sensor. The antenna will dissipate only a few tens of Watts while it is activated. Under AC excitation conditions, the inductive reactance will add to the impedance, moreso, the higher the drive frequency, reducing the component of current in phase with the voltage and hence reducing the power required to drive the antenna. The ME-33 element will generate several hundreds of Volts (depending on its size as well as the circuit and device it drives). The ME device is essentially a capacitor that has a value of C typically in the range of 0.1-10 nF. Thus the power stored on the capacitor under the action of a magnetic field alternating at the resonance frequency of the ME sensor (typically 40-80 kH for cm-scale devices) will be in the range of tens of Watts. The power that can be drawn from the ME-33 element is estimated to be hundreds of mW based on the efficiency of these devices, and can be used immediately or stored in a small implanted battery.

The field generated normal to the pancake coil 83 in FIG. 4 (or by the coils in FIGS. 13-14), as a function of distance b from the coil along its normal, is readily calculated. Here l is the current, A is the loop antenna area, πa², and b is the distance from the antenna in cm. FIG. 11 illustrates in graph 66 a decrease in field strength H in Oe-per-ampére with distance b in cm along the axis of symmetry of a 3-cm-diameter coil. At a distance equal to the coil radius, H is about ⅓^{rd} of its value at b=0.

In various applications, the coil, the AC circuit powering it, the configuration of the ME-33 receiver and its power-conditioning circuit can each be modified to optimally meet the implant power needs. For example, for pain relief by nerve stimulation, a very short pulse of high voltage at low current is needed. In this case, more current should be provided by the external power source and the coil should contain more turns of low-resistance wire to increase the field generated.

It should be noted that more power can be harvested by a ME sensor from the external field if the field is applied at the resonance frequency of the sensor. For g₃₃ devices that are symmetric about their mid-plane (no bending modes), the lowest frequency resonance is due to a longitudinal, standing acoustic wave between the electrodes. This mode occurs at a frequency close to

$$f = \frac{1}{2 L} \sqrt{\frac{E}{ρ}} ,$$

where L is the distance between the electrodes, E is the effective modulus of the device and p is the average mass density of the device. For ME sensors that we have made on the cm scale, the resonance frequency is typically tens of kHz.

### Electrical Stimulation of Bone Growth

In accordance with one embodiment of the invention, there is provided a minimally-invasive mode of electrical stimulation for bone growth. This new mode of electrical bone stimulation allows implanted electrodes to provide accurately-targeted therapy at low power, without implanting a large battery so that the implantation can be done with less trauma. At the same time, the system does not require the patient to wear an external battery that needs daily replacement. Instead, a smaller, rechargeable cell is implanted with the electrodes; it is recharged from outside the body by a novel and rapid method of wireless power transfer. Because of the efficiency of the new method of wireless power transfer, a large external apparatus need not be worn continually. Instead a smaller apparatus (a magnetic field transmitter) that wirelessly charges the implanted secondary (rechargeable) battery requires only a few minutes of application to deliver e.g., a few days or a week of continuous electrotherapy. This frees the patient from the need for continuous use of an external apparatus, e.g., strapped about a limb where a bone fracture occurred.

The wireless power transmission system consists of two, or in some cases four, components. The first component is an external magnetic field source such as i) an electrically conducting coil (with or without a magnetic core), through which an AC current flows, or ii) another ME device driven by an AC voltage so that the magnetization in its magnetic layer oscillates, producing an AC magnetic field. In either case the field source should generate a magnetic field peak strength near the fracture site of e.g., 1 kA/m to 2 kA/m at a frequency in the range 1 kHz to 500 kHz and preferably in the range 50 kHz to 300 kHz. The second component is a power receiver consisting of a laminate of magnetostrictive and electroactive materials that convert the alternating magnetic field to an AC voltage. If the power received is not used directly to stimulate bone growth, then a third component, an integrated circuit, is provided to convert the alternating voltage from the ME receiver into a regulated DC voltage that can be used directly for stimulation of bone growth or used to charge an implanted secondary battery (which is the fourth component).

In accordance with one embodiment, energy is transferred wirelessly across 3 cm at a rate of more than 0.25 W, for an implanted ME device that is 0.1 cm³ in volume. The external field that is projected into the body falls below conservative limits for low-frequency magnetic field exposure for the general public; these limits may be exceeded for therapeutic purposes. The integrated circuit implanted with the receiver conditions the energy and delivers electrical energy to the storage device(s) in the body (batteries or capacitors). The electrical energy can be delivered as a high voltage, low current stimulus for promotion of bone growth (or other medical therapy such as nerve stimulation).

The following Table 2 compares broad ranges of power and energy storage capacity typical for each prior art therapy device, and for one embodiment of the invention. It is assumed in each case that the electric field at the fracture site is 1 V/mm across a 1 mm fracture and the current between the electrodes is 20 μA. In the non-invasive case, the electrode spacing is assumed to be 10 cm.

TABLE 2

| | | State-of- | |
|---|---|---|---|
| | | the-art | Minimally-invasive, |
| | Non-invasive | Invasive | present invention |
| Required voltage (V) | 8-80 | 1-8 | 1-8 |
| Power for 20 μA (mW) | 0.16-1.6 | 0.02-0.16 | 0.02-0.16 |
| Battery lifetime | 1 day | 9 months | 1 week |
| Battery capacity (W-h) | 0.004-0.04 | 0.13-1.0 | 0.003-0.02 |

Based on Table 2, column 3, a 3 mW-hr (milli Watt-hour) rechargeable cell could be recharged for 1 week of use in bone growth therapy if a 0.1 cm³ ME receiver were implanted and exposed to a field of 10 Oe for about one min. It may be advantageous to use an even smaller capacity rechargeable cell, such as a thin film rechargeable cell, to minimize the space needed for the implanted system. The much smaller cell could still be charged in a matter of minutes or at most hours to provide electrical bone growth stimulation for a week.

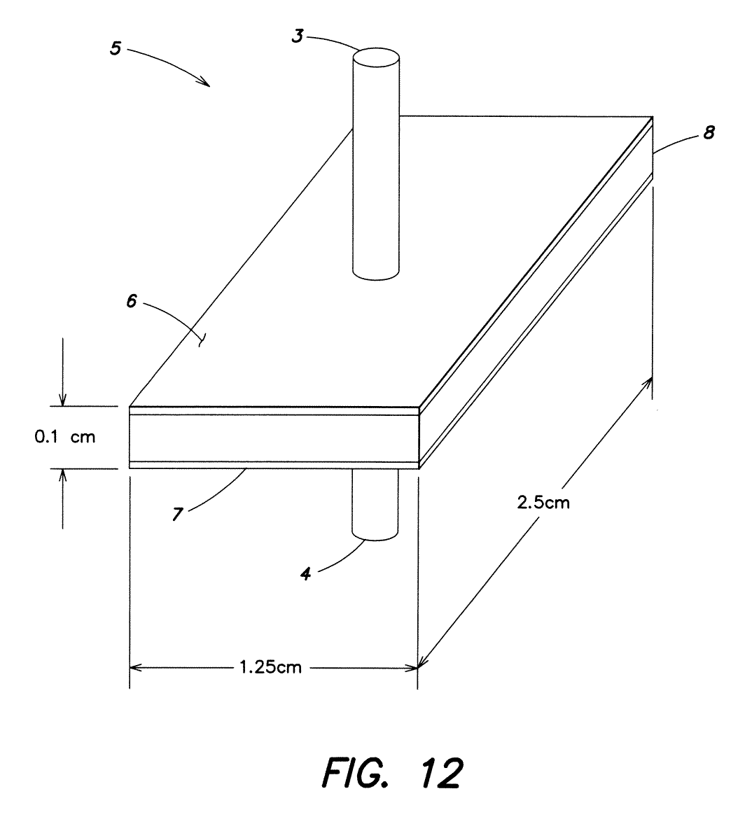

FIG. 12 illustrates one embodiment of an implantable ME receiver 5 comprising two outer amorphous magnetic layers 6, 7 (each 30 μm in thickness), sandwiching a 190 μm thick ceramic PZT piezoelectric layer 8. The device 5 is a generally flat, rectangular article of dimension 2.5 cm (length)×1.25 cm (width)×0.1 cm (height). The two electrodes 3, 4 shown protruding from the large faces of the device can be replaced by thin or thick film electrodes coplanar with the ME element.

FIGS. 13-14 illustrate two types of coils 9, 12 that could be used to produce an AC magnetic field at the ME receiver site near a bone fracture in order to wirelessly generate the power needed to stimulate bone growth. The external source may be a series of small flat coils embedded in an applicator (e.g., a band) that can be wrapped around the limb containing the fractured bone. Alternatively, it may be a pad containing one or more flat coils, to be placed over or adjacent to the affected limb or body part in which the fracture is to be treated. The field source may be of the form shown in FIG. 13, that is, a source 9 including a coil 10 filled with a magnetic core 11; the magnetic field direction is suggested by the curved lines 15. Alternatively, the field source 12 may be a coil 14 without a core, as shown in FIG. 14. As used herein, “coil” is not meant to be limiting and includes any configuration for generating a charging magnetic field (also referred to as an antenna). As previously indicated, an ME device may also be driven to produce a magnetic field and used as the external source. The coil may be configured so that it wraps around the affected limb with its poles on opposing sides of the limb, so that its magnetic field is strongest at the part of the limb between the two pole pieces.

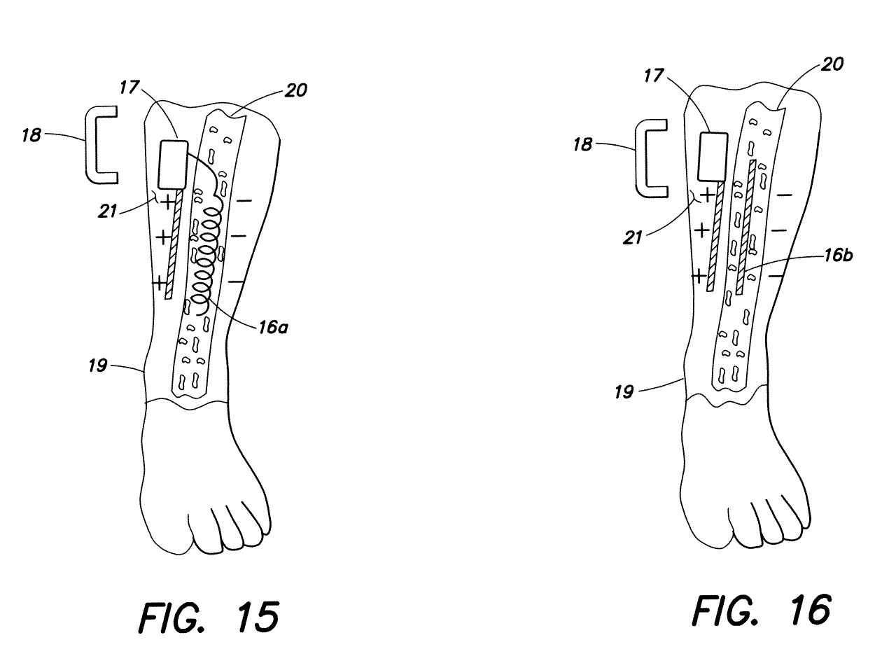

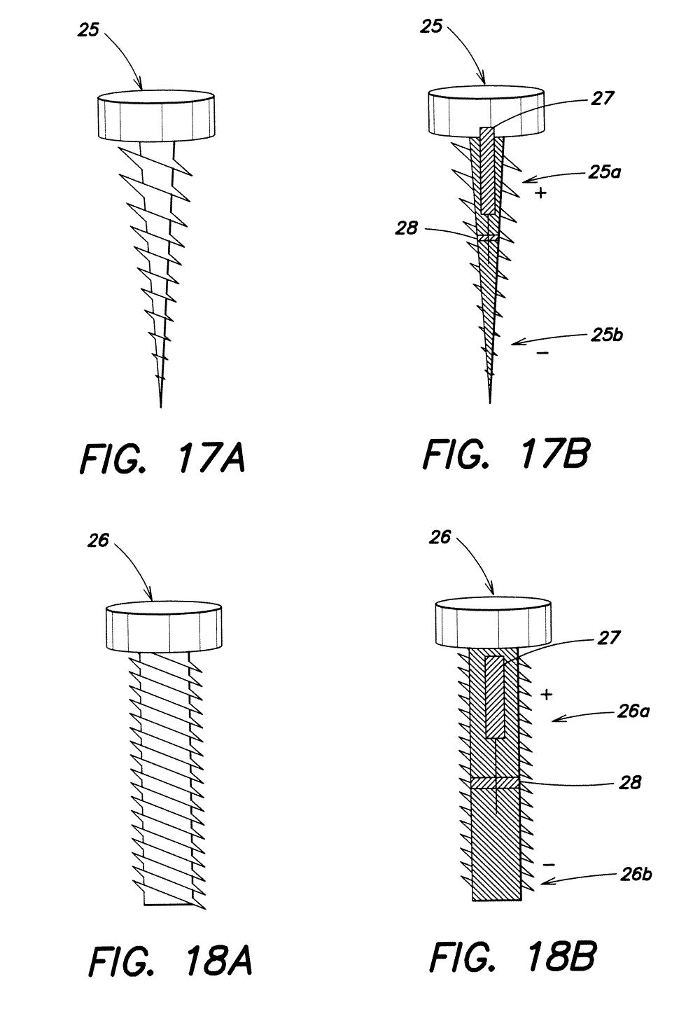

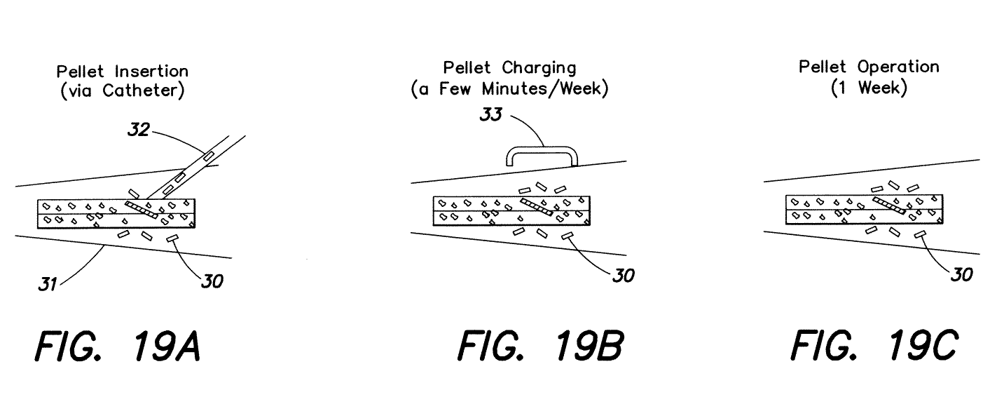

Once the electrical power is available in the body it can be used to enable any of several new embodiments of electrical stimulation of bone growth that are highly localized to the fracture region. These modalities of enhanced bone growth include the following: 1) a coiled electrode, but instead of the prior art long-term (6-9 months) implanted primary cell, a much smaller 1-10 day secondary cell is provided and implanted with the wireless power receiver (see FIG. 15); or 2) a stabilizing rod inserted into the bone core (currently used in some severe fractures) may also act as an electrode in conjunction with a second outer electrode positioned over the outer surface of the bone (see FIG. 16); or 3) electric dipole screws (see FIG. 17) or bolts (see FIG. 18) may be used for securing bone across a fracture line while also providing a local electric field to stimulate bone growth (see FIGS. 17-18); or 4) a multiplicity of small (bean-sized) electric dipole receivers (that include a storage cell) may be implanted via a catheter near the fracture site (see FIG. 19). Each of these alternative embodiments is described in more detail below. In each case, the electric dipole field can optionally be created by using two different ME receivers that are responsive at different frequencies (e.g., of different sizes or materials). The two ME receivers may be connected to an anode and cathode if DC therapy is desired. The voltage applied to the anode ME receiver may oscillate at fₐ between 0 volts and +V₀ volts, while the voltage applied to the cathode ME receiver may oscillate at f_{c} between 0 volts and −V₀ volts.

In the first embodiment shown in FIG. 15, an internally disposed (within the bone 20) coil-like electrode 16a is connected to one electrode of a laminated magneto-electric power receiver 17, the receiver being implanted in soft tissue 21 near the fracture site. The implant 17 includes the ME receiver integrated with a power conditioning chip and a short-term (possibly 1 week) rechargeable cell. Whereas a prior art primary storage cell can typically last up to 9 months, continuously delivering 20 μA at about 1 V, it has a capacity of a few tenths of a Watt-hour (W-h). In contrast, the rechargeable cell used in the present embodiment need only supply about 20 μW for a week, so the rechargeable cell (in implant 17) need only have a capacity of a few milli Watt-hours (mW-h); as a result it can be about 1% of the volume of the prior art implanted cell. The rechargeable cell of the present embodiment has a rate of energy transfer of about 0.3 W using safe AC magnetic field levels; it will take approximately 1 minute to fully charge this cell for a week of continuous therapy. An external charger 18 is illustrated in FIG. 15, positioned over the patient's shin adjacent the embedded receiver 17. This mode of electrotherapy could be applied to vertebral fractures to bring about union as an alternative to cementing to insure immobility at the fracture site.

In the second embodiment shown in FIG. 16, a stabilizing rod 16b implanted in the bone to maintain alignment and provide stabilization during healing also functions an electrode (instead of coil electrode 16a of FIG. 15). The statements made above for FIG. 15, with the exception of the last statement regarding use for vertebral fracture, also apply to FIG. 16. However, because (in FIG. 16) there may be a larger area between the electrodes (based on the length of the fracture), a larger voltage may need to be applied to the electrodes to maintain a constant charge on the electrodes and E field strength at the fracture (see Eq. 1).

In regard to the third embodiment (FIGS. 17-18), in cases of severe bone fracture there are often metal fastener(s) placed in the bone, or a metal clamp connecting two stabilizing braces along the length and on opposite sides of the bone. The metal fastener(s), e.g., a screw 25 or bolt 26, can be manufactured in two electrically-insulated parts (25a, 25b and 26a, 26b, respectively) and contain a power receiver 27 whose two output electrical terminals are connected to the two parts of the fastener to provide an electric field. A power conditioning circuit may be designed to deliver either an AC or DC voltage to the two electrodes, depending on the medically determined optimal therapy.

In regard to the fourth embodiment (FIGS. 19a-c) one or more mm-sized power receivers are carried by one or more pellets 30 which may be implanted near the bone fracture site 31. The pellet(s) 30 may be inserted into the body via a minimally-invasive catheter 32 as shown in FIG. 19a. Each pellet contains a small chip to process the electrical energy generated by the ME element and store it for several days of continuous therapy. Each of the multiple ME pellet receivers may optionally have a different characteristic (resonance), frequency at which to receive electrical power from the external magnetic-field transmitter. Thus each pellet can be individually activated to more sharply focus the therapy from outside the body. Each pellet preferably includes a small chip that processes the received electrical power, converting it to an appropriate DC voltage as needed. The medical practitioner can selectively activate different receivers and observe fluoroscopically their location under activation in order to choose the ones most favorably located to promote healing of the fracture. FIG. 19b shows an external charger 33 which may be positioned adjacent the embedded pellets 30 periodically, e.g., for a few minutes a week, to recharge the pellets. The patient is then free of the external apparatus (charger) as shown in FIG. 19c, for a week, which provides substantial benefits to the patient and will likely increase compliance with the prescribed therapy.

Thus, the benefits of the above-described modes of electrical stimulation of bone growth may include one or more of:
1) Increased patient compliance due to shorter time required for use of external apparatus.
2) Reduced power demands by use of implanted electrodes.
3) Reduced power demands by use of multiple implanted ME receivers;
4) Improved healing/therapeutic results by use of multiple selectively excitable ME receivers for a highly focused therapy.
5) Reduced size of implanted components (e.g., a smaller ME device and a smaller rechargeable cell, or the use of small pellets that contain receiver, power conditioning and storage).
6) Reduced cost, complexity and/or trauma of implant insertion due to smaller size of implant, or insertion of pellets via catheter.

Although exemplary embodiments of the invention have been disclosed, it will be apparent to those skilled in the art that various changes and modifications can be made which will achieve all or some of the advantages of the invention.

The disclosures of all of the following articles and publications is hereby incorporated by reference herein:
U.S. patent application: “Novel, high sensitivity, passive magnetic field sensor”, Jiankang Huang and Robert C. O'Handley, Filing date: provisional, Dec. 9, 2002, Formal, Dec. 8, 2003, Application No. 60/431,487.
U.S. Pat. No. 6,984,902 B1. Jan. 10, 2006, “Novel, high efficiency, vibration energy harvester”, Jiankang Huang, Robert C. O'Handley, and D. Bono. Filing date: provisional, Feb. 3, 2003, Formal, Jan. 29, 2004. Application No. 60/444,562.
“New, high-sensitivity, hybrid magnetostrictive/electroactive magnetic field sensors”, Jingkang Huang, R. C. O'Handley and D. Bono, SPIE Conf. Proc., 5050, 229 (2003).
“Passive”, solid-state magnetic field sensors and applications thereof,” Yi Qun Li, R. C. O'Handley and G. Dionne, U.S. Pat. No. 6,279,406 B1, issued Aug. 28, 2001.
“High magnetoelectric properties in 0.68 Pb(Mg_{1/3}Nb_{2/3})O₃ 0.32PbTiO₃ single crystal and Terenol-D laminate composites”, J. Ryu et al., J. Korean Ceramic Soc 39, 813, (2002).
“Magnetoelectric properties in piezoelectric and magnetostrictive laminate composites” J. Ryu et al, Jpn. J. Appl. Phys 40, 4948 (2001).
Multilayered, unipoled Piezoelectric Transformers”, S. Priya et al, Jpn. J. Appl. Phys. 43, 3503 (2004).
“Enhanced magnetoelectric effects in laminate composites of Terfenol-D/Pb(Zr,Ti)O₃ under resonant drive”, S. Dong et al. Appl. Phys. Lett. 83, 4812 (2003).
“Longitudinal and transverse magnetoelectric voltage coefficients of magnetostricitve/piezoelectric laminate composite: Theory”, S. Dong et al. IEEE Trans. Ultrasonics, ferroelectrics and Freq. control 50, 1253 (2003).
“Magnetoelectric coupling in Terfenol-D/polyvinylidene-difluoride composites” K. Mori and M. Wuttig, J. Appl. Phys. 81, 100 (2002).
“Stimulating Treatment”, E. Lyle Cain, Jr. M.D., Orthopedic Technology Review 4, July/August 2004.
E. Fukada and I. Yasuda, “On the piezoelectric effect of bone”, J. Phys. Soc. Jpn 12, 1158-1162 (1957).
“Osteoclasts and Ostoeblasts migrate in opposite direction in response to a constant electrical field”, J. Ferrier, S. M. Ross, J. Kanehisa, and J. E. Aubin, Cell Physiol. 129, 283-288 (1986).
“Pulsed electromagnetic fields for treatment of bone fractures: S. A. Satter, M. S. Islam, K. S. Rabbani, and M. S. Talukder, Bangladesh Med. Res. Counc. Bull. 25, 6-10 (1999).
T. Schubert, J. Kieditzsch, and I. Wolf, “Results of fluorescence microscopy studies of bone healing by direct stimulation with bipolar impulse currents and with an interference current procedure in the same animal experiment”, Z. Orthop. Ihre Grensgeb. 124, 6-12 (1986).
T. Hanaoka, “The effects of pulsed micro-electrical currents on internal remodeling in long tubular bone and bone healing”, Nippon Seikeigeka Gakkai Zasshi 57, 151-166 (1983).
“Apparatus and method of utilizing a magnetic field”, Jiankang Huang, Hariharan Sundram, Robert C. O'Handley, and David Bono, U.S. patent application Ser. No. 11/734,181, filed Apr. 13, 2007.
“Improved wireless, transcutaneous power transmission for in vivo applications”, R. C. O'Handley, J. K. Huang, David Bono and Jesse Simon, IEEE Sensors, 8, 57-62 (2007).

1. An apparatus comprising:
an external source for transmitting a changing magnetic field into a human or other animal body; and
an implantable magnetostrictive-electroactive (ME) device that converts the changing magnetic field to generate electrical energy for use in the body.

2. The apparatus of claim 1, including:
an implantable storage device coupled to the ME device for storing the electrical energy.

3. The apparatus of claim 2, wherein:
the ME device generates AC electrical energy and the apparatus includes a circuit that converts the AC electrical energy to DC electrical energy for immediate use and/or storage in the storage device.

4. The apparatus of claim 1, wherein:
the external source provides an adjustable magnetic field to adapt the electrical energy for a desired therapy.

5. The apparatus of claim 1, wherein:
the implanted ME device includes a circuit for conditioning the electrical energy for use in the body.

6. The apparatus of claim 2, wherein:
the implanted ME device includes a circuit for conditioning the electrical energy.

7. The apparatus of claim 1, wherein:
the external source comprises a coil driven by an AC current.

8. The apparatus of claims 1, wherein:
the external source comprises an applicator including one or more coils for positioning on the body.

9. The apparatus of claim 1, wherein:
the source comprises a coil surrounding a magnetic core.

10. The apparatus of claim 1, wherein:
the external source comprises a ME transmitter.

11. The apparatus of claim 1, wherein:
the storage device comprises a rechargeable cell or capacitor.

12. The apparatus of claim 1, wherein:
the apparatus includes a stabilizing rod, clamp or plate positionable within or in contact with a bone and comprising an electrode.

13. The apparatus of claim 2, wherein:
the apparatus including electrodes driven by the generated or stored electrical energy to produce an electric field in the body.

14. The apparatus of claim 1, wherein:
the electrical energy comprises an electric field adapted to promote bone growth.

15. The apparatus of claim 1, wherein:
the apparatus includes a pellet insertable in the body via a cannula or catheter, and the ME device is carried by the pellet.

16. The apparatus of claim 15, wherein:
the pellet includes a circuit to process the generated electrical energy for storage in the storage device.

17. The apparatus of claim 15, wherein:
the apparatus includes a plurality of such pellets, each carrying an ME device.

18. The apparatus of claim 17, wherein:
the pellets have different resonant frequencies.

19. The apparatus of claim 15, wherein:
the pellet includes two electrodes on an outside surface of the pellet.

20. A method comprising:
providing a magnetostrictive-electroactive (ME) device and an electrical storage device inside a human or other animal body;
transmitting a changing magnetic field into the body;
the ME device converting the changing magnetic field to generate electrical energy; and
using the generated electrical energy for therapy in the body and/or storing the electrical energy in the storage device.

21. The method of claim 20, wherein:
the generated and/or stored energy is used to produce an electric field in the body.

22. The method of claim 20, wherein:
the generated and/or stored energy is used for electrical stimulation of bone growth.

23. The method of claim 20, including:
the ME device generates AC electrical energy; and
converting the AC electrical energy to DC electrical energy for storage in the storage device.

24. The method of claim 20, including:
providing electrodes in the body and driving the electrodes with the generated and/or stored energy.

25. The method of claim 24, including:
providing one or more of the electrodes in or in contact with bone to provide electrical stimulation of bone growth.

26. The method of claim 24, including:
implanting one electrode in a bone and providing another electrode on an outer surface of a bone.

27. The method of claim 24, wherein:
the electrodes are positioned across a bone fracture.

28. The method of claim 27, wherein:
a spacing between the electrodes is in a range from 0.2 mm to 20 mm.

29. The method of claim 20, including:
generating an electrical current density in a range of 10 μA to 20 mA.

30. The method of claim 20, including:
generating an electrical current density in a range of 10 μA to 100 μA.

31. The method of claim 20, including:
using the generated and/or stored energy to deliver an electric field at a bone fracture site.

32. The method of claim 31, including:
providing a magnetic field peak strength at the fracture site in a range of 10 A/m to 2 kA/m.

33. The method of claim 31, including:
providing a magnetic field peak strength at the fracture site in a range of 80 A/m to 2 kA/m.

34. The method of claim 31, wherein:
the magnetic field having a frequency in a range of 30 kHz to 500 kHz.

35. The method of claim 20, wherein:
the storage device having a capacity on the order of kilowatt-hours.

36. The method of claim 20, wherein:
the storage device having a capacity of 1 milliWatt-hours to 20 kiloWatt-hours.

37. The method of claim 20, wherein:
the storage device having a capacity of 100 Watt-hours to 20 kiloWatt-hours.

38. The method of claim 20, including:
recharging the storage device by storing the generated energy.

39. The method of claim 36, wherein:
the storage device can be fully recharged in 30 minutes or less.

40. The method of claim 20, wherein:
delivering the generated and/or stored energy at transfer rate of 10 milliWatts to 1 Watt.

41. The method of claim 20, including:
delivering the generated and/or stored energy as a high voltage, low current signal for promotion of bone growth.

42. The method of claim 20, wherein:
the storage device has a capacity to deliver electrical energy for a given medical therapy for 10 days or less.

43. The method of claim 20, including:
providing an external source of the changing magnetic field on an outer surface of the body.

44. The method of claim 43, wherein:
the source comprising one or more coils wrapped around a limb of the body.

45. The method of claim 43, wherein:
the source comprising one or more coils placed against the skin.
